(19) United States
(12) Patent Application Publication
(10) Pub. No.: US 2024/0082584 A1
(43) Pub. Date: Mar. 14, 2024

(54) PACING CAPTURE VERIFICATION IN WEARABLE MEDICAL SYSTEM

(71) Applicant: Kestra Medical Technologies Inc., Kirkland, WA (US)

(72) Inventor: Jaeho Kim, Kirkland, WA (US)

(21) Appl. No.: 18/313,220

(22) Filed: May 5, 2023

Related U.S. Application Data
(60) Provisional application No. 63/406,219, filed on Sep. 13, 2022.

(51) Int. Cl. A61N 1/37; A61B 5/00; A61B 5/363; A61N 1/362; A61N 1/365

(52) U.S. Cl. CPC A61N 1/371; A61B 5/363; A61B 5/746; A61N 1/3625; A61N 1/365

(57) ABSTRACT

A wearable medical system delivers and verifies capture of pacing pulses. The wearable medical system includes a support structure, a plurality of ECG electrodes, an energy output device coupled to an output circuit, a processor, and a plurality of therapy electrodes. The processor is in communication with the plurality of ECG electrodes and the output circuit. The processor causes a pacing pulse to be delivered to the patient via the energy output device, the output circuit, and the plurality of therapy electrodes. The processor determines whether the pacing pulse was captured by determining whether the ECG signal, within a window subsequent to a blanking period and a refractory period following delivery of the pacing pulse, meets one or more capture criteria. In response to a determination that the pacing pulse was captured, delivery of an additional pacing pulse is prevented else an additional pacing pulse is delivered to the patient.

### CROSS-REFERENCE TO RELATED APPLICATIONS

This application claims priority to and the benefit of the provisional patent application No. 63/406,219 titled “Pacing Capture Verification In Wearable Medical Device,” filed in the United States Patent and Trademark Office on Sep. 13, 2022. The specification of the above referenced patent application is incorporated herein by reference in its entirety.

### TECHNICAL FIELD

The present technology relates to a wearable medical system and more particularly, but not by way of limiting, the present technology relates to providing pacing therapy to patients and verifying pacing capture using the wearable medical system.

### BACKGROUND

Cardiac rhythm disorders or cardiac conditions occur when electrical signals that coordinate heart's beats are awry or faulty. Such faulty signaling causes the heart to either beat too fast, too slow, and/or irregularly. A cardiac rhythm disorder corresponding to a fast, abnormal heart rhythm, that starts in the lower chambers of the heart, is known as Ventricular Tachyarrhythmia (VT). Another cardiac rhythm disorder corresponding to an irregular heart rhythm where the lower heart chambers contract in a very rapid and uncoordinated manner is known as Ventricular Fibrillation (VF). A cardiac rhythm disorder corresponding to slower heart beats is known as bradyarrhythmia and a cardiac rhythm disorder corresponding to a cessation of electrical and mechanical activity of the heart is known as asystole. There is a higher possibility that at least one of the cardiac rhythm disorders leads to Sudden Cardiac Arrest (SCA) that endangers the life of a patient. Early detection and therapy for the cardiac rhythm disorders, such as VF, may prevent the resultant disastrous situations.

Conventionally, arrhythmia detectors are utilized for detecting the cardiac rhythm disorders such as VT and VF. Further, even if the arrhythmia detector detects the cardiac rhythm disorders, additional devices are required for providing therapy to treat the underlying condition. In some situations, due to faulty electrodes attached to the patient or existing environmental factors, the arrhythmia detector erroneously determines the presence of the cardiac rhythm disorder. In another scenario, where the arrhythmia detectors accurately identify the cardiac rhythm disorder, a device providing the therapy would provide the therapy without determining if the therapy was successful. In a situation where the therapy was a failure, other types of resuscitation attempts may have to be applied. However, due to the lack of determination of failure or success of the therapy, the patient may be exposed to life-threatening situations.

### SUMMARY

The present disclosure relates to a wearable medical system for delivering and verifying the capture of pacing pulses. In one aspect of the present disclosure, the wearable medical system comprises a support structure, a plurality of Electrocardiogram (ECG) electrodes to sense an ECG signal of a patient, and an energy output device to store an electrical charge. The energy output device may be a capacitor. The plurality of ECG electrodes may be resistive, DC-coupled ECG electrodes. The wearable medical system further comprises an output circuit coupled to the energy output device, a plurality of therapy electrodes, and a processor. The plurality of therapy electrodes is engaged to the support structure and in communication with the output circuit, to deliver therapy to the patient. The processor is in communication with the plurality of ECG electrodes and the output circuit.

The processor is configured to cause a pacing pulse to be delivered to the patient via the energy output device, the output circuit, and the plurality of therapy electrodes. The processor determines whether the pacing pulse was captured by determining whether the ECG signal, within a window subsequent to a blanking period and a refractory period following delivery of the pacing pulse, meets one or more capture criteria. The blanking period is implemented digitally using a digital filter such as a finite impulse response filter, after delivering the pacing pulse for a finite time. The digital filter continues filtering ECG signals during an electrical artifact period. In response to a determination that the pacing pulse was captured, delivery of an additional pacing pulse to the patient is prevented until another pacing interval is expired. In response to a determination that the pacing pulse was not captured, an additional pacing pulse is caused to be delivered to the patient. A user interface is configured to alert bystanders through audio and/or visual alerts of at least one of: a pacing pulse is being delivered to the patient and a pacing capture status.

### BRIEF DESCRIPTION OF THE DRAWINGS

The above-mentioned implementations are further described herein with reference to the accompanying figures. It should be noted that the description and figures relate to exemplary implementations and should not be construed as a limitation to the present disclosure. It is also to be understood that various arrangements may be devised that, although not explicitly described or shown herein, embody the principles of the present disclosure. Moreover, all statements herein reciting principles, aspects, and embodiments of the present disclosure, as well as specific examples, are intended to encompass equivalents thereof.

FIG. 1 illustrates an example of a wearable medical system (WMS) worn by a user, according to an embodiment of the present disclosure.

FIG. 2 illustrates a conceptual diagram with multiple electrodes of the WMS, according to an embodiment of the present disclosure.

FIG. 3 illustrates a block diagram of an external pacer and defibrillator, according to an embodiment of the present disclosure.

FIG. 4 illustrates a capture scenario, according to an embodiment of the present disclosure.

FIG. 5 illustrates a no-capture scenario, according to an embodiment of the present disclosure.

FIG. 6 illustrates a graph depicting an application of a finite impulse response (FIR) filter on an Electrocardiogram (ECG) signal, according to an embodiment of the present disclosure.

FIG. 7 illustrates a graph with waveforms from ECG electrodes, according to an embodiment of the present disclosure.

FIG. 8 illustrates an example method for delivering and verifying the capture of pacing pulses using the WMS, according to an embodiment of the present disclosure.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, and the like. In other instances, well-known structures or methods, associated with a wearable medical system, have not been shown or described in detail to avoid unnecessarily obscuring descriptions of the embodiments.

Unless the context indicates otherwise, throughout the specification and claims which follow, the word “comprise” and variations thereof, such as, “comprises” and “comprising” are to be construed in an open, inclusive sense that is as “including, but not limited to.” Further, the terms “first,” “second,” and similar indicators of the sequence are to be construed as interchangeable unless the context clearly dictates otherwise.

Reference throughout this specification to “one aspect” or “an aspect” means that a particular feature, structure, or characteristic described in connection with an embodiment is included in at least one aspect. Thus, the appearances of the phrases “in one aspect” or “in an aspect” in various places throughout this specification are not necessarily all referring to the same embodiment. Furthermore, the particular features, structures, or characteristics may be combined in any suitable manner in one or more aspects.

As used in this specification and the appended claims, the singular forms “a,” “an,” and “the” include plural referents unless the content clearly dictates otherwise. It should also be noted that the term “or” is generally employed in its broadest sense, that is, as meaning “and/or” unless the content clearly dictates otherwise.

The present disclosure relates to the wearable medical system that detects cardiac rhythm disorders of a patient and provides one or more therapeutic electrical pulses to the patient based on the detected cardiac rhythm disorder. The wearable medical system also verifies if the electrical pulses were captured by the heart of the patient.

In certain aspects, the wearable medical system provides alerts, such as audible alerts, when providing the electrical pulses to the patient for alerting the patient and bystanders in contact with the patient or in close proximity to the patient. The wearable medical device also provides alerts related to status of the capture of the electrical pulses by the heart of the patient. Upon providing an alert that the electrical pulses were not captured, a bystander or an emergency medical technician, communicatively coupled to the wearable medical system, is notified of a no-capture situation. The notification allows the bystander or an emergency medical technician to determine or execute a next course of action and communicates a health status of the patient, thereby reducing exposure of the patient to life-threatening situations.

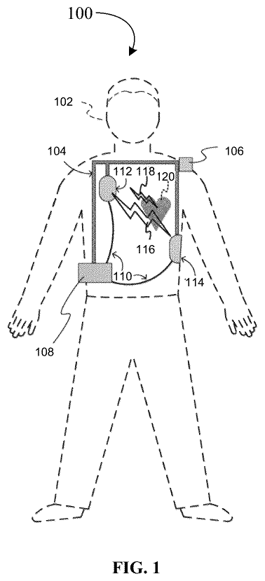

FIG. 1 illustrates a wearable medical system (WMS) 100 worn by a patient 102, according to an embodiment of the present disclosure. Depending on the context, the patient 102 may also be referred to as a person 102 and/or a wearer 102, since the patient 102 is wearing components of the WMS 100, or as a user 102 using the WMS 100. The patient 102 could be ambulatory, that is, while wearing the WMS 100, the patient 102 can walk around and is not necessarily bed ridden. While the patient 102 may also be considered to be a “user” of the WMS 100, this definition is not exclusive to the patient 102. For instance, the user 102 of the WMS 100 may also be a clinician such as a doctor, nurse, emergency medical technician (EMT), or other similarly tasked individual or group of individuals. In some cases, the user 102 may even be a bystander. The particular context of these and other related terms within this description should be interpreted accordingly. The WMS 100 at least includes one or more components such as a support structure 104, an outside monitoring device 106, an external pacer and defibrillator (EPD) 108, and electrode leads 110 that allow coupling of defibrillation electrodes 112 and 114 to the EPD 108.

The support structure 104 may be configured to be worn by the patient 102 for at least several hours per day, during the night, one or more days, and/or one or more months. The support structure 104 may be implemented in many different ways. For example, the support structure 104 may be implemented in a single component or a combination of multiple components. In some embodiments, the support structure 104 may include a vest, a half-vest, a garment, or the like, such that the support structure 104 may be worn similarly to analogous articles of clothing. In some embodiments, the support structure 104 may include a harness, one or more belts or straps, and the like. In some embodiments, the support structure 104 may be worn by the patient 102 around the torso, hips, over the shoulder, and the like. In some embodiments, the support structure 104 includes a container or housing that may be waterproof. Further, the support structure 104, in some embodiments, may be worn by being attached to the patient's body by an adhesive material, for example as shown and described in U.S. Pat. No. 8,024,037. The support structure 104 may be implemented as a support structure described in U.S. Patent Publication No. US 2017/0056682 A1, which is incorporated herein by reference. The person skilled in the art will recognize that the components of the WMS 100 may be in the housing of the support structure 104 instead of being attached externally to the support structure 104, for example as described in the aforementioned '682 document. It shall be understood that the support structure 104 is shown generically in FIG. 1 and merely illustrates concepts about the support structure 104. FIG. 1 is not to be construed as limiting with respect to either a manner in which the support structure 104 is implemented or how the support structure 104 is worn. Also, the support structure 104 may be implemented in various other examples.

The WMS 100, according to some embodiments, may obtain data from the patient 102 which is referred to as patient data. For collecting the patient data, the WMS 100 may, in some embodiments, include at least the outside monitoring device 106, also referred to as a device 106 hereinafter. The device 106 may be provided as a standalone device, for example, external to the EPD 108. The device 106 may be configured to sense or monitor one or more local parameters. The one or more local parameters may be one or more parameters of the patient 102, one or more parameters of the WMS 100, or one or more parameters of the environment, without limitation.

The device 106 may include one or more sensors or transducers for obtaining the one or more parameters. Each of the one or more sensors may be configured to sense the one or more parameters of the patient 102, the WMS 100, and/or the environment. Each of the one or more sensors are further configured to render an input responsive to the sensed one or more parameters. In some embodiments, the rendered input is quantitative, such as values of a sensed parameter. In some embodiments, the input is qualitative, such as indicating whether one or more thresholds are crossed, and the like. In some embodiments, the rendered inputs about the patient 102 are also called physiological inputs or patient inputs. In some embodiments, a sensor may be construed more broadly, as encompassing more than one individual sensor.

In some embodiments, the device 106 is physically coupled to the support structure 104. Additionally, the device 106 may be communicatively coupled with other components that are coupled to the support structure 104. The communication between the device 106 and the other components may be implemented by a communication module, as will be deemed applicable by a person skilled in the art in view of this description.

The EPD 108 is also referred to as a pacer 108 or as a main electronics module 108. A component of the EPD 108 may be configured to store electrical charges. Other components may cause at least some of the stored electrical charges to be discharged via the defibrillation electrodes 112 and 114, for delivering electrical pulses to the patient 102. The EPD 108 may initiate defibrillation, hold-off defibrillation, or initiate pacing, based on a combination of a variety of inputs, with an Electrocardiogram (ECG) signal merely being one of the varieties of inputs.

The defibrillation electrodes 112 and 114 are also referred to as electrotherapy electrodes 112 and 114 or therapy electrodes 112 and 114. The defibrillation electrodes 112 and 114 may be configured to be positioned on the body of the patient 102 in a number of ways. For instance, the EPD 108 and the defibrillation electrodes 112 and 114 may be coupled to the support structure 104, directly or indirectly. In an example, the support structure 104 may be configured to be worn by the ambulatory patient 102 to maintain at least one of the defibrillation electrodes 112 and 114 on the body of the patient 102, while the patient 102 is moving around. The defibrillation electrodes 112 and 114 may be thus maintained on the body of the patient 102 by being attached to the skin of the patient 102, such that the defibrillation electrodes 112 and 114 are pressed against the skin directly or through the garment, and the like, of the patient 102.

In some embodiments, the defibrillation electrodes 112 and 114 are not necessarily pressed against the skin but may become biased upon sensing a condition that may merit intervention by the WMS 100. Additionally, some of the components of the EPD 108 may be considered coupled to the support structure 104 directly, or indirectly via at least one of the defibrillation electrodes 112 and 114.

The electrical pulses may be categorized based on energy of the electrical pulses. The electrical pulses are categorized as defibrillation shock 116 and one or more pacing pulses 118 that are typically much lower in energy than the defibrillation shock 116. The action of delivering the defibrillation shock 116 is also called shocking the patient 102, and the action of delivering the one or more pacing pulses 118 is called pacing. The one or more pacing pulses 118 are intended to pace the heart 120 if needed, and typically the one or more pacing pulses 118 are caused to be delivered in a periodic sequence by appropriately timed discharges. The defibrillation shock 116 is also referred to as cardioversion shock, therapy shock, or the like. The electrical pulses corresponding to the defibrillation shock 116 may also be referred to as defibrillation pulses 116. In accordance with the embodiments of the present disclosure, the EPD 108 may also include one or more modules to detect whether the delivery of the one or more pacing pulses 118 resulted in a capture by the heart 120 of the patient 102.

When the defibrillation electrodes 112 and 114 make good electrical contact with the body of the patient 102, the EPD 108 may administer one or more brief electric pulses to the body of the patient 102, such as the defibrillation shock 116 or the one or more pacing pulses 118 via the defibrillation electrodes 112 and 114. The administration of the defibrillation shock 116 or the one or more pacing pulses 118, based on corresponding requirement, is referred to as electrotherapy. The defibrillation shock 116 or the one or more pacing pulses 118 have attributes suitable for their purpose.

The defibrillation shock 116 is typically stronger than the one or more pacing pulses 118 such that the defibrillation shock 116 may have an energy of at least 100 Joules (J), for example, 200 J, 300 J, 360 J, and the like. The defibrillation shock 116 is intended to go through and restart the heart 120 of the patient 102, in an effort to save the life of the patient 102.

The one or more pacing pulses 118 are not intended to be administered concurrently with the defibrillation shock 116. The one or more pacing pulses 118 are depicted to be smaller than the defibrillation shock 116 to reflect that the one or more pacing pulses 118 have less energy than the defibrillation shock 116, for example, less than 30 Joules. In some embodiments, the one or more pacing pulses 118 are a discharge from at least the two electrodes, which are either the same defibrillation electrodes 112 and 114 used for the defibrillation shock 116, or different therapy electrodes (not shown).

The WMS 100 implements transcutaneous pacing for which consideration of one or more aspects is necessary for the pacing delivery. In an example, an impedance of the defibrillation electrodes 112 and 114 may vary based on the patient 102, such as dryness or moisture of the skin, the manner in which the defibrillation electrodes 112 and 114 contact the skin, location of placement of the defibrillation electrodes 112 and 114, and the like. Further, energy delivered by the defibrillation electrodes 112 and 114 may also vary, for example, the energy delivered depends on the impedance that may be variable. Also, relative positioning of the heart 120 with respect to the defibrillation electrodes 112 and 114 may also cause ECG amplitude to vary, which in turn may complicate a rhythm analysis of the ECG signal.

In some scenarios, if the patient 102 is experiencing fine VF, low amplitude of the fine VF may be interpreted as asystole and may cause initiation of external pacing. Further, upon pacing due to the fine VF, the pacing will not likely result in capture, which in turn may cause the energy of the pacing to be increased, in an effort to get the capture. The external pacing is painful to the patient 102, hence notifying or alerting an Emergency Medical Services (EMS) personnel or the EMT may be appropriate rather than evaluating one or more thresholds corresponding to the external pacing. Further, during the application of the external pacing, alerting bystanders or any medical personnel regarding the application of external pacing may be beneficial.

In some embodiments, the one or more components of the WMS 100 may be customized for the patient 102. The customization may include one or more aspects, such as providing the support structure 104 that is custom-fit for the body of the patient 102. Further, baseline physiological parameters of the patient 102 may be measured for various scenarios, such as when the patient 102 is lying down (in various orientations), sitting, standing, walking, running, and or the like. The baseline physiological parameters may include heart rate of the patient 102, motion detector outputs, one for each scenario, and the like. Values of the measured baseline physiological parameters may be used to customize the WMS 100, to make accurate diagnoses for the patient 102. The customization of the WMS 100 allows other patients with bodies different from one another to use the WMS 100. Values of the measured baseline physiological parameters may be stored in a memory of the WMS 100, and so on. A programming interface, in some embodiments, receives the measured values of the baseline physiological parameters. The programming interface may provide an input related to the measured values of the baseline physiological parameters to the WMS 100 automatically, along with other data.

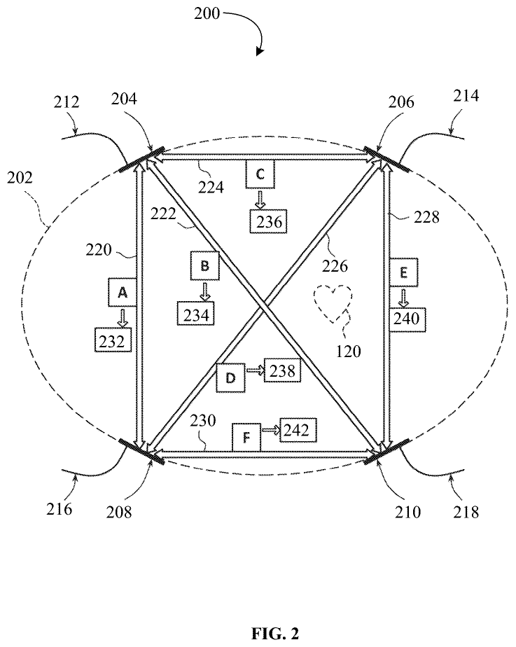

FIG. 2 illustrates a conceptual diagram 200 illustrating a section 202 of the patient 102 with multiple ECG electrodes 204, 206, 208, and 210 of the WMS 100 positioned around the heart 120 of the patient 102. FIG. 2 is described in conjunction with the previous figure. The ECG electrodes 204, 206, 208, and 210 are also referred to as ECG sensing electrodes 204, 206, 208, and 210, and collectively referred to as ECG electrodes 204-210. The patient 102 is viewed from the top and the patient 102 is facing downwards. The section 202 is obtained when a plane intersects the patient 102 at the torso. The ECG electrodes 204, 206, 208, and 210 may be maintained on or surround the torso of the patient 102, and have respective wire leads 212, 214, 216, and 218. The ECG electrical potentials that may be measured at the ECG electrodes 204, 206, 208, and 210 have values E1, E2, E3, E4.

Any pair of the ECG electrodes 204, 206, 208, and 210 defines a vector, along which an ECG signal may be sensed and/or measured. The ECG electrodes 204, 206, 208, and 210 pairwise define vectors 220, 222, 224, 226, 228, and 230, thereby illustrating a multi-vector embodiment. In some embodiments, although the ECG electrodes 204, 206, 208, and 210, and the vectors 220, 222, 224, 226, 228, and 230 are illustrated, other number of ECG electrodes and/or vectors may be implemented. In some embodiments, all the vectors 220, 222, 224, 226, 228, and 230 may not be considered. For example, the vectors 224 and 230 may be ignored since the vectors 224 and 230 least traverse the torso of patient 102 compared to other vectors such as the vectors 220, 222, 226, and 228.

It will be understood that the ECG electrodes 204, 206, 208, and 210 are illustrated to be shown on a same plane for simplicity of explanation. However, the ECG electrodes 204, 206, 208, and 210 may not necessarily exist on the same plane. Consequently, the vectors 220, 222, 224, 226, 228, and 230 may not necessarily exist on the same plane either. The vectors 220, 222, 224, 226, 228, and 230, collectively referred to as vectors 220-230, define channels A, B, C, D, E, and F, respectively. ECG signals 232, 234, 236, 238, 240, and 242 may thus be sensed and/or measured from the channels A, B, C, D, E, and F, respectively, and particularly from the appropriate pairings of the wire leads 212, 214, 216, and 218 for each channel. The ECG signals 232, 234, 236, 238, 240, and 242, also collectively referred to as ECG signals 232-242, may or may not be sensed concurrently.

The above-mentioned formalism renders values of the ECG signals 232-242 that are sensed between pairs of the ECG electrodes 204, 206, 208, and 210 using the vectors 220, 222, 224, 226, 228, and 230. For example, the ECG signal 232 at channel A has a voltage E1−E2=E12. In some embodiments, a different formalism is utilized for deriving ECG signal values for each of the ECG electrodes 204, 206, 208, and 210 by itself, and at a corresponding location and not necessarily in a pair with another ECG electrode.

The different formalism includes considering a point at a virtual position (not shown) between the four ECG electrodes 204, 206, 208, and 210 within the torso of the patient 102. An average ECG voltage value (CM) may be ascribed to that point. The CM is derived from a statistic of voltages at the ECG electrodes 204, 206, 208, and 210. The virtual position continuously changes based on voltages of the ECG electrodes 204, 206, 208, and 210. However, an actual sensor for sensing the voltage at that point is ignored or not considered. Nevertheless, the different formalism further considers a virtual main central terminal (MCT) (not shown), which would sense the CM. In the different formalism, therefore, the vectors 220, 222, 224, 226, 228, and 230 are considered from each of the ECG electrodes 204, 206, 208, and 210 to the MCT. Relative to the MCT, there may be four resulting vectors with values of the corresponding signals, which may be considered as: E1C=E1−CM, E2C=E2−CM, E3C=E3−CM, and E4C=E4−CM. In some embodiments, the vectors 220, 222, 224, 226, 228, and 230 are formed in software by selecting a pair of the signals and subtracting one from the other. For example, E1C−E2C=(E1−CM)−(E2−CM)=E1−E2+(CM−CM)=E1−E2=E12.

Thus, by having the multiple channels A, B, C, D, E, and F, the WMS 100 may assess which one of the multiple channels A, B, C, D, E, and F provides the best ECG signal for capture analysis. Alternatively, instead of just one channel, the WMS 100 may determine to keep two or more, but not all, of the channels and use the corresponding ECG signals for the capture analysis, for instance as described in U.S. Pat. No. 9,757,581, issued on Aug. 23, 2017.

In some embodiments, the WMS 100 may be implemented with multiple ECG electrodes, beyond the ECG electrodes 204, 206, 208, and 210 to generate multiple vectors (or channels) for monitoring the rhythm of the heart 120 of the patient 102. The WMS 100 continuously monitors the corresponding ECG signals 232-242 of the patient 102 to detect the cardiac rhythm disorders and may also monitor, in a further enhancement, activity of the patient 102 for noise detection. The WMS 100 may also include the therapy electrodes 112 and 114, such as the defibrillation electrodes 112 and 114, for delivering the transcutaneous pacing pulses, such as the one or more pacing pulses 118, in response to detection of bradycardia/asystole. The use of multiple vectors may help in an improved and accurate capture analysis that includes determination of the capture of the delivered one or more pacing pulses 118.

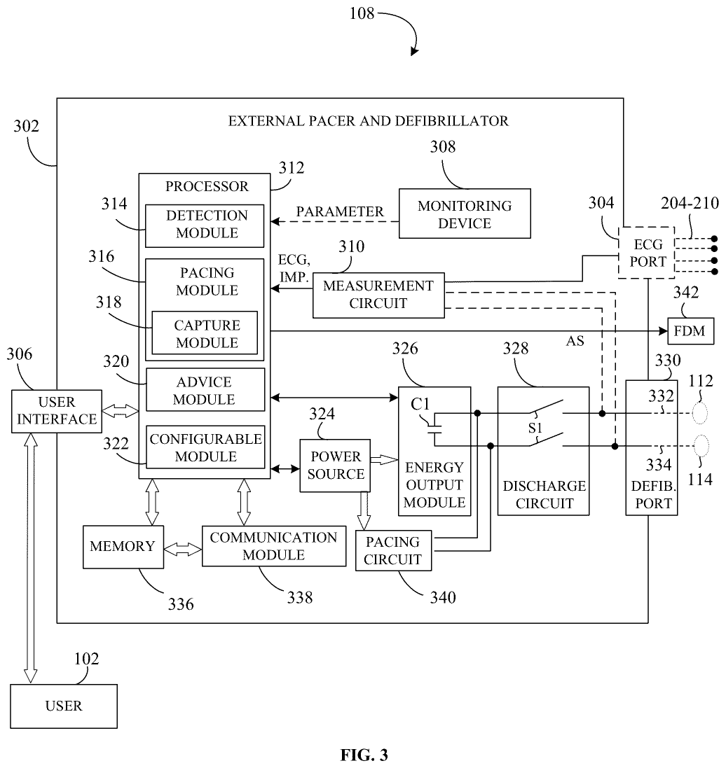

FIG. 3 illustrates the external pacer and defibrillator (EPD) 108 of the WMS 100 that is capable of providing the pacing to the patient 102, according to an embodiment of the present disclosure. FIG. 3 is described in conjunction with the previous figures. The EPD 108 is also capable of verifying capture of the one or more pacing pulses 118, or the pacing with pacing capture detection. In some embodiments, the EPD 108 is intended for the patient 102 who may be carrying it on their body, such as the ambulatory patient 102.

One or more components of the EPD 108 are stored in a housing 302, which may also be referred to as a casing 302. The EPD 108 at least includes components such as an ECG port 304, a user interface 306, a monitoring device 308, a measurement circuit 310, a processor 312, a power source 324, an energy output module 326, a discharge circuit 328, a defibrillation port 330 coupled to the defibrillation electrodes 112 and 114, a memory 336, a communication module 338, a pacing circuit 340, and a fluid deploying mechanism 342. The terms “external pacer and defibrillator (EPD),” and “wearable medical system (WMS),” are interchangeably used since the WMS 100 includes EPD 108 unless the context clearly dictates otherwise.

The user interface 306 may include one or more output devices, which may be visual, audible, audio, or tactile, for communicating with the user 102 by outputting images, sounds, or vibrations. The communicated output perceivable by the patient 102 or the user 102 may also be called human-perceptible indications (HPIs). The HPIs may be used to alert the patient 102, provide sound alarms that may be intended also for bystanders, and the like. For example, an output device of the one or more output devices may be a light that may be turned on and off, a screen to display sensed, detected, and/or measured information by the WMS 100 and provide visual feedback to a rescuer, such as the user 102, for resuscitation attempts, and the like. Another output device of the one or more output devices may be a speaker, which may be configured to issue voice prompts, alerts, beeps, loud alarm sounds and/or words, and the like. The output provided by the one or more output devices may be communicated to the user 102, such as the bystander, when defibrillating or pacing, and so on.

The user interface 306 may further include one or more input devices for receiving inputs from the user 102, such as the patient, the local trained caregiver, the bystander, and the like. In some embodiments, the user 102 may be a local rescuer at a scene, such as the bystander who might offer assistance, or a trained person. In some embodiments, the user 102 may be a remotely located trained caregiver in communication with the WMS 100. The one or more input devices may include various controls, such as push buttons, keyboards, touchscreens, one or more microphones, and the like.

One of the one or more input devices may be a cancel switch, also referred to as an “I am alive” switch or “live man” switch. Actuating the cancel switch, for example, may prevent the impending delivery of the defibrillation shock 116, or the one or more pacing pulses 118, to the patient 102. In some embodiments, the output device such as the speaker may be configured to output a warning prompt prior to the impending or planned defibrillation shock 116 or pacing sequence of the one or more pacing pulses 118 being caused to be delivered. The cancel switch is configured to be actuated by the patient 102 in response to the warning prompt.

The impending or planned defibrillation shock 116 or pacing sequence of the one or more pacing pulses 118 is caused to halt responsive to the actuation of the cancel switch after the warning prompt has been output. Operations of the processor 312 and methods may include causing the speaker to output the warning prompt and determining whether or not the cancel switch has been actuated after the warning prompt has been output.

The ECG port 304, also referred to as a sensor port 304, is coupled to or adapted for plugging in one or more of the ECG electrodes 204-210. The ECG electrodes 204-210, in some embodiments, are resistive, DC-coupled ECG electrodes. The ECG electrodes 204-210, for example, may be connected continuously to the ECG port 304. In an example, an impedance of the ECG electrodes 204-210 may vary based on the patient 102, such as dryness or moisture of the skin, a manner in which the ECG electrodes 204-210 contact the skin, location of placement of the ECG electrodes 204-210, and the like.

The ECG electrodes 204-210 are types of transducers that may sense an ECG signal, for example, a 12-lead signal. In some embodiments, the ECG electrodes 204-210 may sense a signal from a different number of leads, especially if the ECG electrodes 204-210 make good electrical contact with the body of the patient 102 and in particular with the skin of the patient 102. The ECG electrodes 204-210 may be attached to the inside of the support structure 104 for making good electrical contact with the patient 102. In some embodiments, the defibrillation electrodes 112 and 114 may be attached to the inside of the support structure 104. The ECG electrodes 204-210 continue to sense the ECG signal during the delivery of the defibrillation shock 116, or the one or more pacing pulses 118.

The WMS 100, according to some embodiments, may also include the fluid deploying mechanism 342 for deploying fluid automatically between the ECG electrodes 204-210 and the skin of the patient 102. The fluid may include an electrolyte, for establishing better electrical contact between the ECG electrodes 204-210 and the skin of the patient 102, thereby making the fluid conductive. When the fluid is deployed, the electrical impedance between the ECG electrodes 204-210 and the skin is reduced. The fluid may be in the form of a low-viscosity gel that does not flow away from the ECG electrodes 204-210 after the fluid has been deployed. The fluid may be used for the defibrillation electrodes 112 and 114 and the ECG electrodes 204-210.

The fluid may be initially stored in a fluid reservoir (not shown), coupled to the fluid deploying mechanism 342 and the support structure 104. The fluid deploying mechanism 342 may be configured to cause at least some of the fluid to be released from the fluid reservoir. The fluid deploying mechanism 342 and/or the fluid reservoir may be deployed near one or more locations to which the ECG electrodes 204-210 and/or the defibrillation electrodes 112 and 114 are configured to be attached to the patient 102. In some embodiments, the fluid deploying mechanism 342 is activated prior to an electrical discharge responsive to receiving an activation signal (AS) from the processor 312.

The monitoring device 308 of the EPD 108 is also referred to as an internal monitoring device 308 since the monitoring device 308 is incorporated within the housing 302. The monitoring device 308 may sense or monitor patient parameters such as physiological parameters of the patient 102, state parameters of the patient 102, system parameters, and/or environmental parameters, all of which may be referred to as patient data. In an example, the monitoring device 308 may include or may be coupled to one or more sensors. In some embodiments, the monitoring device 308 may be complementary or an alternative to the outside monitoring device 106. Allocating which patient parameters are to be monitored by the monitoring device 308 and the device 106 may be determined according to design considerations.

The physiological parameters of the patient 102, for example and without limitation, include one or more physiological parameters data that may assist the EPD 108 in detecting whether or not the patient 102 needs a shock, other intervention, or assistance. The physiological parameters may also, in an example, include physiological parameters data such as medical history of the patient 102, event history, and the like. The physiological parameters data may further include the ECG signal values, blood oxygen level, blood flow, blood pressure, blood perfusion, pulsatile change in light transmission or reflection properties of perfused tissue, heart sounds, heart wall motion, respiration-related information, breathing sounds, and pulse of the patient 102.

Accordingly, the monitoring device 308 and/or the outside monitoring device 106 may include one or more sensors configured to acquire patient physiological signals. In some embodiments, the one or more sensors or transducers may include the one or more ECG electrodes 204-210 to detect or obtain the ECG signals 232-242, a perfusion sensor, a pulse oximeter, a device for detecting blood flow, for example, a Doppler device, and the like. In some embodiments, the one or more sensors may include a sensor for detecting blood pressure, for example, a cuff, an optical sensor, illumination detectors, and the one or more sensors perhaps working together with light sources for detecting a color change in a tissue. In some embodiments, the one or more sensors may include a motion sensor, a device that may detect the heart wall motion or movement, a sound sensor, a device with a microphone, a SpO2 sensor, and the like. In view of this disclosure, it will be appreciated that such sensors may help detect the pulse of the patient 102, and may therefore also be called pulse detection sensors, pulse sensors, or pulse rate sensors. In addition, a person skilled in the art may implement other ways of performing pulse detection.

In some embodiments, the monitoring device 308, the outside monitoring device 106, and/or the processor 312 may detect a trend in the monitored physiological parameters data of the patient 102. The trend may be detected by comparing values of parameters at different times over short and/or long terms. The physiological parameters whose detected trends may help a cardiac rehabilitation program include a) cardiac function, for example, ejection fraction, stroke volume, cardiac output, and the like; b) heart rate variability at rest or during exercise; c) heart rate profile during exercise and measurement of activity vigor, such as from the profile of an accelerometer signal and informed from adaptive rate pacemaker technology; d) heart rate trending; e) perfusions, such as from SpO2, CO2, or other parameters such as those mentioned above; f) respiratory function, respiratory rate, and the like; g) motion, level of activity; and other similar parameters.

The detected trend may be stored and/or reported via one or more wired or wireless communication links, along with a warning if warranted, to a physician monitoring progress or health status of the patient 102. The reported trends provide clarity and updated information corresponding to the patient 102, to the physician. The physician may gauge a condition that is either not improving or deteriorating based on the reported trends.

The state parameters may include recorded aspects of the patient 102, such as but not limited to the motion, posture, whether the patient 102 has spoken or communicated with a physician recently along with what has been spoken, and the like. In some embodiments, the state parameters may further include a history of the state parameters. In an example, the monitoring device 308 may include a location sensor such as a Global Positioning System (GPS) location sensor. The location sensor may detect the location of the patient 102, and speed may be detected as a rate of change of location over time.

In some embodiments, the monitoring device 308 may include the motion detectors that may be configured to detect a motion event and output a motion signal indicative of motion of the motion detectors, and thus the motion of the patient 102. The state parameters may assist in narrowing down the determination of whether Sudden Cardiac Arrest (SCA) is indeed occurring. In some embodiments, the WMS 100 may include the motion detectors. The motion detectors may be implemented in many ways as known in the art, for example, by using an accelerometer. The motion event may be defined as convenient, such as a change in motion from a baseline motion or rest, and the like. In an example, the motion detectors are implemented within the monitoring device 308. In response to the detected motion event, the motion detectors may render or generate a motion detection input that may be received by a subsequent device or functionality.

The system parameters of the WMS 100 may include system identification, battery status, system date and time, reports of self-testing, records of data entered, records of episodes and interventions, and the like. The environmental parameters may include ambient temperature and pressure. Moreover, a humidity sensor may provide information as to whether or not it is likely raining. The detected location of the patient 102 may also be considered as one of the environmental parameters. The patient's location may be presumed or considered, if the monitoring device 308 or the outside monitoring device 106 includes the GPS location sensor as mentioned above, and if the patient 102 is wearing the WMS 100.

The EPD 108 may also include the measurement circuit 310 that may be communicatively coupled to the sensors/transducers and the monitoring device 308, in some embodiments. The measurement circuit 310 may be configured to sense one or more electrical physiological signals of the patient 102 from the sensor port 304. In some embodiments, if the EPD 108 lacks the sensor port 304, the measurement circuit 310 may, in an example, obtain physiological signals through nodes 332 and 334 instead, when the defibrillation electrodes 112 and 114 are attached to the patient 102. The input to the measurement circuit 310 through the nodes 332 and 334 is an ECG signal that reflects the ECG measurement. The patient data, in an example, may be the ECG signals 232-242 that may be sensed as a voltage difference between the defibrillation electrodes 112 and 114. In addition, the patient parameters may include an impedance, which may be sensed between the defibrillation electrodes 112 and 114 and/or between the connections of the sensor port 304 considered pairwise.

Sensing the impedance may be useful for detecting, among other processes, whether the defibrillation electrodes 112 and 114 and/or the sensing electrodes 204-210 are making good electrical contact with the body of the patient 102. The patient's physiological signals may be sensed when available. The measurement circuit 310 may render or generate information about the patient's physiological signals as inputs, data, other signals, and the like. As such, the measurement circuit 310 may be configured to render the patient inputs responsive to the patient parameters sensed by the one or more sensors. In some embodiments, the measurement circuit 310 may be configured to render the patient inputs, such as values of the ECG signals 232, 234, 236, 238, 240, and 242, responsive to the ECG signals 232, 234, 236, 238, 240, and 242 sensed by the ECG electrodes 204-210. Information rendered by the measurement circuit 310 is an output, however, output information may be called an input because the information is received as an input by a subsequent device or functionality.

The EPD 108 also may include the processor 312 that may be implemented in different ways in various embodiments. The different ways include, by way of example and not of limitation, digital and/or analog processors such as microprocessors and Digital Signal Processors (DSPs), controllers such as microcontrollers, software running in a machine, programmable circuits such as Field Programmable Gate Arrays (FPGAs), Field-Programmable Analog Arrays (FPAAs), Programmable Logic Devices (PLDs), Application Specific Integrated Circuits (ASICs), any combination thereof, and the like. In some embodiments, the processor 312 may be implemented using multiple electronic devices distributed in various parts of the EPD 108.

The processor 312 may include, or have access to, a non-transitory storage medium, such as the memory 336 that, in some embodiments, is a non-volatile component for storage of machine-readable and machine-executable instructions. A set of such instructions can also be called a program. The instructions, which may also be referred to as “software,” generally provide functionality by performing acts, operations, and/or methods as may be disclosed herein or understood by one skilled in the art in view of the disclosed embodiments. In some embodiments, and as a matter of convention used herein, instances of the software may be referred to as a “module” and by other similar terms. Generally, a module includes a set of instructions, to offer or fulfill a particular functionality and the processor 312 includes one or more modules. Embodiments of modules and the functionality delivered are not limited by the embodiments described in this document.

In some embodiments, the processor 312 includes a plurality of modules such as a detection module 314, a pacing module 316, an advice module 320, and a configurable module 322. The detection module 314, for example, may include a Ventricular Fibrillation (VF) detector. At least one ECG signal of the ECG signals 232-242 sensed through the ECG electrodes 204-210 may be received as data by the detection module 314 from the measurement circuit 310. The data to the detection module 314 may be available as inputs or data that reflect values, or values of other signals, may be used by the VF detector to determine whether the patient 102 is experiencing VF. Detecting the VF is useful since the VF typically results in the SCA. The detection module 314 may also include a Ventricular Tachycardia (VT) detector, and/or a bradycardia/asystole detector for detecting the bradycardia and/or asystole, and the like. The detection module 314 is also referred to as a cardiac condition detector 314. In some embodiments, a QRS detector, included in the detection module 314, may run on a single vector of the vectors 220-230. However, if multiple vectors corresponding to the vectors 220-230 are used, then a designated QRS detector may be assigned to each of the multiple vectors.

The advice module 320, in some embodiments, may receive an output of the detection module 314 and generate advice for the one or more components of the EPD 108 regarding a subsequent course of action. The advice module 320 may provide a variety of advice based on the output of the detection module 314. In some embodiments, the advice is a shock or no shock determination to the processor 312. The shock or no shock determination may be made by executing a stored shock advisory algorithm. The shock advisory algorithm can, according to some embodiments, make a shock or no shock determination from the ECG signals 232-242 that are obtained using the ECG electrodes 204-210, and determine whether or not a shock criterion is met. The determination may be made from the rhythm analysis of the obtained ECG signals 232-242 or otherwise. For example, there may be shock decisions for the VF, VT, and the like.

In some embodiments, the advice module 320 utilizes the sensed and monitored patient parameters along with the stored shock advisory algorithm and/or the rhythm analysis of the captured ECG signals 232-242 for making the shock or no shock determination. In some embodiments, the advice module 320 may also provide inputs to the one or more output devices to indicate or notify the patient 102 regarding the impending delivery of the defibrillation shock 116 or the one or more pacing pulses 118, upon determining that the shock has to be provided. The advice module 320 may be capable of receiving an input from the patient 102, via the cancel switch, for aborting the delivery of the defibrillation shock 116 or the one or more pacing pulses 118. If the patient 102 determines an erroneous detection of a cardiac rhythm disorder, upon receiving at least a notification regarding the impending delivery of the defibrillation shock 116 or delivery of the pacing pulses 118, then the patient 102 provides the input to abort the delivery of the defibrillation shock 116 or the one or more pacing pulses 118.

In some embodiments, when the one or more pacing pulses 118 have to be delivered and/or are being delivered to the patient 102, the WMS 100 may alert the bystanders and/or medical personnel that the WMS 100 is delivering the transcutaneous pacing. The alerts may also provide a pacing capture status that indicates whether the one or more pacing pulses 118 were captured by the patient 102. The pacing module 316 may control the user interface 306 to output the alerts. The alerts may help the bystander 102 to avoid interfering with the delivery of pacing, as well as preventing the bystander from experiencing painful transcutaneous pacing if the bystander is physically in contact with the patient 102 while the pacing is being delivered. The alerts may also help emergency responders, such as the EMS personnel, by informing the emergency responders of the pacing. Further, the EPD 108 may alert or notify the EMS personnel whether the one or more pacing pulses 118 were captured and the energy level or current level of the one or more pacing pulses 118. The information provided to the EMS personnel may help the EMS personnel in deciding the next course of action.

The initiation of the pacing, in some embodiments, may begin with the advice module 320 determining that the cardiac rhythm disorder is the bradycardia or asystole condition. The advice module 320 may then indicate the pacing module 316 to perform the pacing. In some embodiments, after receiving the advice for the defibrillation, the pacing module 316 may deliver the defibrillation shock 116 to the patient 102. In some embodiments, after receiving the advice for the pacing, the pacing module 316 may consecutively search for an interval to deliver the one or more pacing pulses 118 to the patient 102, where the interval to deliver the one or more pacing pulses 118 is also referred to as a pacing interval. In some embodiments, the pacing module 316 is activated to provide and control the one or more pacing pulses 118 based on the determination of the cardiac rhythm disorder regardless of input or the advice from the advice module 320. The ECG signals 232-242 utilized for the determination of the cardiac rhythm disorder are referred to as a first set of ECG signals 232-242. In some embodiments, the first set of ECG signals may include at least one of the ECG signals 232-242.

A pacing pulse, of the one or more pacing pulses 118, delivered to the patient 102 immediately after receiving the advice is referred to as a first pacing pulse 118. The pacing module 316 may implement and/or control the ability of the EPD 108 to pace the heart 120. The ability of the pacing module 316 to pace is referred to as a pacing capability. In response to detecting an arrhythmia that can be treated with the pacing, for example, the VF, the pacing module 316 may initiate a delivery process of the one or more pacing pulses 118. For example, the pacing module 316 controls the power source 324, the energy output module 326, and/or the discharge circuit 328 to output the one or more pacing pulses 118. In embodiments that include the pacing circuit 340, the pacing module 316 may control the pacing circuit 340 and the discharge circuit 328 to output the one or more pacing pulses 118 to the patient 102.

In some embodiments, when the advice module 320 determines to shock the patient 102, the one or more electrical pulses are delivered to the patient 102 by the EPD 108, through at least one of the pacing circuit 340 or the energy output module 326. Delivering the one or more electrical pulses is also known as discharging, shocking the patient 102 for defibrillation, pacing, and the like. In ideal conditions, a reliable shock or no shock determination may be made by analyzing a segment of at least one ECG signal of the detected ECG signals 232-242 of the patient 102. In practice, however, the ECG signals 232-242 are often corrupted by electrical noise, which reduces the accuracy of the analyses of the ECG signals 232-242 and results in an incorrect detection of cardiac rhythm disorder, leading to a false alarm to the patient 102. Noisy ECG signals may be handled as described in U.S. patent application Ser. No. 16/037,990, filed on Jul. 17, 2018 and published as US 2019/0030351 A1, and also in U.S. patent application Ser. No. 16/038,007, filed on Jul. 17, 2018 and published as US 2019/0030352 A1, both by the same applicant and each is incorporated herein by reference.

The processor 312 may include additional modules, such as the configurable module 322 that, in some embodiments, is specifically coupled to an accelerometer. Several movements of the patient 102 may result in higher heart rate which may be erroneously considered as the tachyarrhythmia condition, thereby resulting in provision of the defibrillation shock 116. By utilizing the accelerometer and the configurable module 322 specifically coupled to the accelerometer, the EPD 108 may determine the current status of the patient 102, such as the movement of the patient 102, with lesser delay and deliver the defibrillation shock 116 or the one or more pacing pulses 118, accordingly, with an increased level of accuracy.

The EPD 108 may also include the power source 324, which is configured to provide an electrical charge in the form of a current or one or more electrical pulses. To enable portability of the EPD 108, the power source 324, in some embodiments, may include a battery. The battery, for example, is a battery pack, which may either be rechargeable or non-rechargeable. In an example, a combination of both the rechargeable and the non-rechargeable battery packs is used. An embodiment of the power source 324 may include an alternate current (AC) power override, from where AC power may be available, an energy-storing capacitor, and the like. Appropriate components may be included to provide for charging or replacing the power source 324. In some embodiments, the power source 324 is controlled and/or monitored by the processor 312.

In some embodiments, the EPD 108 may further include the energy output module 326, which is also referred to as an energy output device 326. The energy output module 326 may be coupled to the support structure 104 either directly or via the defibrillation electrodes 112 and 114 and the respective electrode leads 110. The energy output module 326 may be coupled to receive the electrical charge provided by the power source 324. The energy output module 326 may be configured to store the electrical charge received by the power source 324. The energy output module 326 temporarily stores electrical energy in form of the electrical charge, when preparing for discharge to administer the defibrillation shock 116 or the one or more pacing pulses 118 to the patient 102. Hence, in some embodiments, the energy output module 326 may be referred to as an energy storage module 326. In some embodiments, the energy output module 326 may be charged from the power source 324 to the desired amount of energy as controlled by the processor 312. The energy output module 326 includes a capacitor C1, which may be a single capacitor or a system of capacitors, and the like. In some embodiments, the energy output module 326 includes a device that exhibits high power density, such as an ultracapacitor. As described above, the capacitor C1 stores the energy in the form of the electrical charge, for delivering the shock, such as the defibrillation shock 116 or the one or more pacing pulses 118, to the patient 102.

A decision to shock may be made responsive to the shock criterion being met, as per the above-mentioned determination. When the decision is to discharge the electrical pulses, the processor 312 may be configured to cause at least some or all of the electrical charge stored in the energy output module 326 to be discharged to the defibrillation electrodes 112 and 114 while the support structure 104 is worn by the patient 102. The discharge of the electrical pulses may include the delivery of the defibrillation shock 116 or the one or more pacing pulses 118 to the patient 102.

For causing the discharge, the EPD 108 may include the discharge circuit 328, also referred to as an output circuit 328. The discharge circuit 328 is coupled to the energy output module 326 and the pacing circuit 340, and in communication with the defibrillation electrodes 112 and 114. If the decision is to provide the defibrillation shock 116, the processor 312 may be configured to control the discharge circuit 328 to discharge at least some of or all of the electrical charge stored in the energy output module 326 in a desired waveform. If the decision is to merely pace, which is to deliver the one or more pacing pulses 118, the processor 312 may be configured to control the discharge circuit 328 to discharge at least some of the electrical charge provided by the power source 324. Since the pacing requires lesser charge and/or energy than the energy or charge for the defibrillation shock 116, in some embodiments, pacing wiring (not shown) is provided from the power source 324 to the discharge circuit 328. The pacing wiring bypasses the energy output module 326. In some embodiments, where solely the pacing is provided with no defibrillation, the energy output module 326 may not be required.

A pacing current may be provided from the power source 324 via the pacing circuit 340, which may be a current source. In some embodiments, the defibrillation shock 116 is delivered using the energy output device 326, and the one or more pacing pulses 118 are delivered using the pacing circuit 340. In some embodiments, the pacing circuit 340 may be omitted since the EPD 108 provides the one or more pacing pulses 118 from the power source 324 and the energy output module 326. In some embodiments, the energy output module 326 is a current source device that provides the one or more pacing pulses 118. In some embodiments, the EPD 108 may include a charger (not shown) that delivers the electrical charge from the battery to the energy output module 326. In some embodiments, the charger may include a charge pump to transfer charge from the battery to the capacitor C1 of the energy output module 326. Either way, the discharging may be performed to the nodes 332 and 334 followed by the defibrillation electrodes 112 and 114 to enable delivery of the defibrillation shock 116 and/or the one or more pacing pulses 118 to the patient 102.

The discharge circuit 328 may include one or more switches 51. The switches 51 may be made or arranged in a number of ways, such as by an H-bridge, and the like. In some embodiments, different switches 51 may be used for a discharge where the defibrillation shock 116 is caused to be delivered, than for a discharge where the weaker one or more pacing pulses 118 are caused to be delivered. The discharge circuit 328 may also be thus controlled via the processor 312, and/or the user interface 306. A time waveform of the discharge may be controlled by controlling the discharge circuit 328. The amount of energy of the discharge may be controlled by how much the energy output module 326 has been charged, and also by how long the discharge circuit 328 is controlled to remain open. In some embodiments, a combination of the power source 324, the energy output module 326, the discharge circuit 328, and the defibrillation port 330 coupled to the defibrillation electrodes 112 and 114 is capable of providing the defibrillation shock 116 and the one or more pacing pulses 118, based on the necessity, as described in U.S. Provisional Patent Application No. 63/420,523, filed on Oct. 28, 2022. In some embodiments, a combination of the power source 324, the pacing circuit 340, the discharge circuit 328, and the defibrillation port 330 coupled to the defibrillation electrodes 112 and 114 is capable of providing the one or more pacing pulses 118, by bypassing the energy output module 326.

The defibrillation port 330 may be a socket in the housing 302, or other equivalent structure. The defibrillation port 330 includes the nodes 332 and 334. Leads of the defibrillation electrodes 112 and 114, such as the electrode leads 110, may be plugged into the defibrillation port 330, to make electrical contact with the nodes 332 and 334, respectively. The defibrillation electrodes 112 and 114 are connected continuously to the defibrillation port 330, instead. Either way, the defibrillation port 330 may be used for guiding, via the defibrillation electrodes 112 and 114, at least some of the electrical charge that has been stored in the energy output module 326 to the patient 102. The electric charge is provided as the shock for defibrillation, pacing, and the like. In some embodiments, the defibrillation shock 116 is delivered synchronously or asynchronously. In some embodiments, the pacing module 316 provides intimation regarding the decision to deliver the one or more pacing pulses 118 and the user interface 306, through the one or more output devices, notifies the user, such as the bystander, that the one or more pacing pulses 118 are being delivered to the patient 102.

The ability of the EPD 108 to pace the heart 120 of the patient 102 may be implemented in a number of ways. The ECG electrodes 204-210 may sense at least one of the ECG signals 232-242 and the processor 312 may measure the sensed ECG signals 232-242 for delivering the pacing pulse 118 after the pacing interval. The pacing interval is a time duration after detection of a QRS complex in the sensed ECG signals 232-242 or after delivering the one or more pacing pulses 118. The pacing may be software controlled, for example, by managing defibrillation path, or by managing the pacing circuit 340, separately, which may output the one or more pacing pulses 118 to the patient 102 via the defibrillation electrodes 112 and 114.

Further, upon delivering the one or more pacing pulses 118, the EPD 108 determines whether the delivered one or more pacing pulses 118 resulted in a capture. For example, the EPD 108 determines whether one of the one or more pacing pulses 118, such as the first pacing pulse 118, induces a QRS complex and/or other necessary artifacts of at least one of the ECG signals 232-242. The capture module 318 of the pacing module 316 may be configured to determine whether the pacing results in the capture. In some embodiments, the capture module 318 monitors the ECG signals 232-242 of the patient 102 for a predefined time window subsequent to a blanking period and a refractory period, determines whether the first pacing pulse 118 was captured, and meets the one or more capture criteria. The predefined time window opens after a predetermined time period, which is also referred to as the blanking period. During the blanking period, the channels A, B, C, D, E, and F corresponding to the ECG electrodes 204-210 are blanked which inhibits the process of sensing the second set of ECG signals 232-242. However, during the blanking period, the ECG electrodes 204-210 continue to be coupled with the EPD 108 or attached to the patient 102. The usage of the ECG electrodes 204-210 such as the DC-coupled ECG electrodes, as described in U.S. patent application Ser. No. 18/073,248, filed on Dec. 1, 2022, and/or a digital filter, for example, a Finite Impulse Response (FIR) filter eliminates a necessity of two sets of electrodes and/or switches.

The refractory period is followed after the blanking period and present in between the artifacts of at least one of the ECG signals 232-242. The blanking period and the refractory period are followed after the delivery of one of the one or more pacing pulses 118, such as the first pacing pulse 118, for determining whether the capture is attained. The ECG signals 232-242 that are monitored during the predefined time window by the capture module 318 for determining the capture are referred to as a second set of ECG signals 232-242. In some embodiments, the second set of ECG signals includes at least one of the ECG signals 232-242.

The second set of ECG signals 232-242 sensed during the predefined time window is compared with the one or more capture criteria to determine whether the capture has occurred. The one or more capture criteria may include the second set of ECG signals 232-242 reaching a predetermined ECG amplitude threshold, and remaining at or above the predetermined amplitude threshold for a predetermined minimum amount of time. The predetermined ECG amplitude threshold is also referred to as a voltage threshold. In response to the determination that the first pacing pulse 118 was successfully captured by the heart 120 of the patient 102, the delivery of additional pacing pulses to the patient 102 is prevented. In some embodiments, after successfully capturing the pacing pulse 118, the subsequent pacing pulse is delivered if no intrinsic QRS is detected for the corresponding pacing interval.

On the other hand, if the capture module 318 determines that the capture is not attained, in some embodiments, the pacing module 316 may provide the additional pacing pulses 118. The additional pacing pulses 118 are provided to the patient 102 after providing the first pacing pulse 118. The delivery of the additional pacing pulses 118 to the patient 102 is prevented until another pacing interval is expired. The additional pacing pulses 118 are also referred to as subsequent pacing pulses 118 or collectively referred to as a second pacing pulse 118.

The second pacing pulse 118 may be discharged to the patient 102 with or without modification in one or more parameters of the subsequent pacing pulses 118 in order to get the capture. The one or more parameters of the one or more pacing pulses 118 may include energy level, current level, and the like.

In some embodiments, if the capture module 318 determines that the pacing does not result in capture, or if the capture is lost after a successful pacing, the pacing module 316 may perform a back-up pacing immediately. The subsequent pacing pulses 118 in the back-up pacing have a maximum pacing energy level. For example, in some embodiments, the maximum pacing energy level of the subsequent pacing pulses 118 may be set at 0.5 J, and in some embodiments, the maximum pacing energy level of the subsequent pacing pulses 118 may be set in the range from about 0.1 J to about 3 J.

In some embodiments, if the capture module 318 determines that the pacing does not result in capture, or if capture is lost after successful pacing, the pacing module 316 may perform incremental pacing. Each of the subsequent pacing pulses 118 in the incremental pacing may have an increment in pacing energy level compared with a previous pacing pulse. In some embodiments, an increment in the pacing energy level is set at 0.1 J. In some embodiments, the increment in the pacing energy level may be set in the range from about 0.05 J to about 0.15 J. In some embodiments, a maximum energy level is set to 0.5 J, but the maximum energy level may be set in a range from about 0.1 J to about 3 J. In some embodiments, the increments in the pacing energy level and the maximum level are defined in terms of current rather than energy. For example, in some embodiments, the current increment may be set at 40 mA and the maximum current level may be set at 200 mA. In the incremental pacing, a preferred amount of electric current which is selected in a range is increased in small increments until the capture is detected. In some embodiments, the incremental pacing may be used to determine the threshold corresponding to the capture. Within the context of the present disclosure, the additional one or more pacing pulses 118 delivered, upon determining that there is no capture of the first pacing pulse 118, are collectively referred to as the second pacing pulse 118. Further, the additional one or more pacing pulses 118 corresponding to the back-up pacing or incremental pacing are referred to as the second pacing pulse 118.

In some embodiments, if the capture module 318 determines that the pacing is not captured, the capture module 318 provides an input to the user interface 306 to notify a status corresponding to the capture to the user 102, such as the bystander, using at least the one or more output devices, described above.

The EPD 108 may include the communication module 338 for establishing the one or more wired or wireless communication links with other devices of other entities, such as a remote assistance center, the EMS, and the like. The communication module 338 may be similar to the communication module disclosed in FIG. 1. The communication links may be used to transfer data and commands. The data may be the patient data, event information, therapy attempted, Cardiopulmonary resuscitation (CPR) performance, system data, environmental data, and so on. The communication links, in some embodiments, may be utilized to transfer the physiological parameters data. For example, the communication module 338 may wirelessly transmit heart rate, respiratory rate, and other vital signs data daily to a server accessible over the internet, for instance as described in U.S. Patent Publication No. US 2014/0043149 A1.

The physician of the patient 102 may directly analyze the communicated data or the communicated data may also be analyzed automatically by algorithms designed to detect a developing illness and then notify the medical personnel via text, email, phone, and the like. The communication module 338 may also include interconnected sub-components which may be deemed necessary by a person skilled in the art, for example but not limited to, an antenna, portions of the processor 312, supporting electronics, outlet for a telephone or a network cable, and the like.

The EPD 108 may further include the memory 336, which is communicatively coupled with the processor 312. The memory 336 may be implemented in a number of ways, such as but not limited to, volatile memories, Non-Volatile Memories (NVM), Read-Only Memories (ROM), Random Access Memories (RAM), magnetic disk storage media, optical storage media, smart cards, flash memory devices, any combination thereof, and the like. The memory 336 stores one or more prompts for the user 102 if the user 102 is a local rescuer. Moreover, the memory 336 may store data including the patient data, for example, as received by the monitoring device 308. The data may be stored in memory 336 before it is transmitted out of the EPD 108, or after the data is received by the EPD 108.

The memory 336 is, thus, a non-transitory storage medium that may include programs for the processor 312, which the processor 312 may be able to read and execute. More particularly, the programs may include sets of instructions in the form of code, which the processor 312 may be able to execute upon reading. The programs may also include other information such as configuration data, profiles, scheduling, and the like, that may be acted upon by the instructions. The execution is performed by physical manipulations of physical quantities, and may result in functions, operations, processes, acts, actions and/or methods to be performed. In some embodiments, the processor 312 is configured to cause other devices or components or blocks to perform functions, operations, processes, acts, actions and/or methods mentioned above. The programs may be operational for the inherent needs of the processor 312 and may also include protocols and to assist the advice module 320 in decision-making.

The non-transitory computer-readable storage medium is encoded or configured to store computer program instructions, which are pacing pulse delivery and verification instructions, defined by modules, for example, 306, 308, 310, 314, 316, 318, 320, 322, 338, 340, and the like, which when executed by a computing device, such as the EPD 108 or the processor 312, cause the computing device to perform operations for delivering and verifying the one or more pacing pulses 118 to the patient 102.

The operations include receiving the first set of the ECG signals 232-242 of the patient 102 via one or more of the ECG electrodes 204-210. The operations further include determining, by the cardiac condition detector 314, if the first set of ECG signals 232-242 are indicative of the cardiac condition or the cardiac rhythm disorders treatable by the pacing. The operations further include activating the energy output device 326, the output circuit 328, and plurality of electrodes, such as the defibrillation electrodes 112 and 114, to deliver the first pacing pulse 118 to the patient 102 upon detecting the cardiac condition such as the bradyarrhythmia condition. The operations further include receiving the second set of ECG signals 232-242 of the patient 102 via the one or more ECG electrodes 204-210 and determining, using the digital filter, the blanking period that begins after the first pacing pulse 118 is delivered for a finite time.

Further, the operations include determining, by the capture module 318, if the second set of ECG signals 232-242 meet the one or more capture criteria indicative of the capture of the first pacing pulse 118 after the blanking period. The capture module 318 identifies the capture of the first pacing pulse 118 when the second set of ECG signals 232-242 exceeds at least one of the amplitude threshold and/or a duration threshold. In response to the determination by the capture module 318 that the second set of ECG signals 232-242 is not indicative of capture of the first pacing pulse 118, the EPD 108 delivers the second pacing pulse 118. The EPD 108 utilizes the energy output device 326, the output circuit 328, and the plurality of therapy electrodes 112 and 114 for delivering the second pacing pulse 118. In some embodiments, the EPD 108 utilizes the pacing circuit 340, the output circuit 328, and the plurality of therapy electrodes 112 and 114 to deliver the second pacing pulse 118. In some embodiments, the second pacing pulse 118 is delivered with the same pacing parameters as the first pacing pulse 118. In some embodiments, the second pacing pulse 118 is a back-up pacing pulse or an incremental pacing pulse. The operations further include activating or utilizing the user interface 306 to generate the audio and/or visual alerts to alert the bystander of the one or more pacing pulses 118 delivered to the patient 102 or the status of the capture.

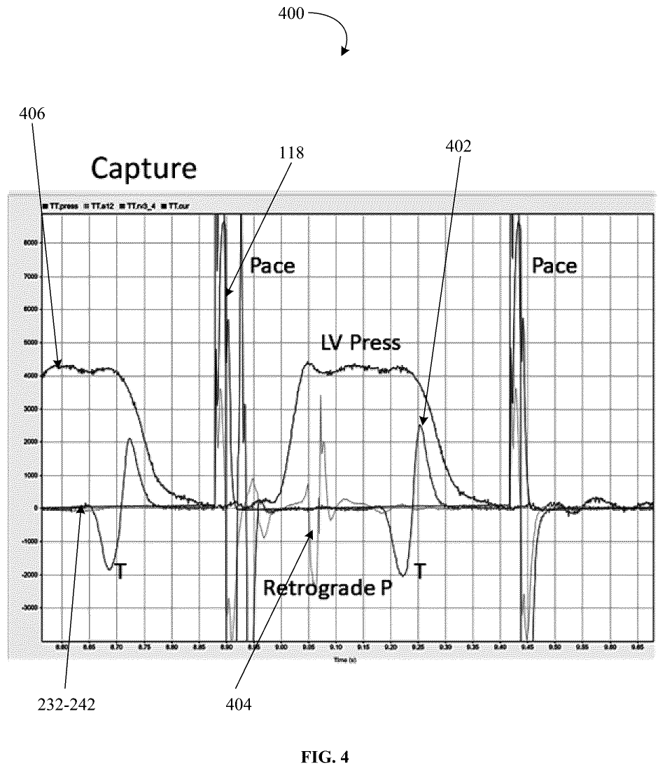

FIG. 4 illustrates a graph 400 depicting a pacing pulse capture scenario, according to an embodiment of the present disclosure. FIG. 4 is described in conjunction with the previous figures. The EPD 108 delivers one of the one or more pacing pulses 118, such as the first pacing pulse 118, upon determining that the patient 102 requires the pacing by the pacing module 316 and/or the advice module 320. The processor 312 delivers the first pacing pulse 118 at a specific instance or phase corresponding to a heartbeat of the heart 120 or the second set of ECG signals 232-242.

A phase in the second set of ECG signals 232-242 between start of a P-wave and end of a QRS complex is referred to as a fusion beat and a phase between the end of the QRS complex and a T-wave is referred to as a refractory period. A phase between the end of the T-wave and the start of the next or consecutive P-wave is referred to as a pacing reception phase, where the heart 120 is receptive to the delivered first pacing pulse 118. The first pacing pulse 118 delivered during the pacing reception phase is considered for determining whether the first pacing pulse 118 resulted in a capture or a no-capture.

The graph 400 depicts the first pacing pulse 118 being delivered to the patient 102 during the pacing reception phase. The capture module 318 performs a pacing pulse capture verification process after the first pacing pulse 118 is delivered to the patient 102. The capture module 318 detects presence or absence of an evoked response (ER) in the sensed second set of ECG signals 232-242 after the first pacing pulse 118 is delivered to the patient 102. The presence of the ER indicates that the pacing resulted in the capture. For example, the capture module 318 determines that the first pacing pulse 118 resulted in the capture when the first pacing pulse 118 immediately induces a wave such as a QRS complex followed by a T-wave as the ER, by monitoring the ECG of the patient 102 for the predefined time window.

The predefined time window opens after the blanking period and after the first pacing pulse 118 is delivered. The capture module 318 senses the second set of ECG signals 232-242 during the predefined time window and compares one or more parameters related to the sensed ECG signals 232-242 to the one or more capture criteria to determine whether the capture has occurred. For example, the one or more capture criteria may include the predetermined amplitude threshold and remaining at or above the predetermined amplitude threshold for the predetermined minimum amount of time. Further, for example, the one or more capture criteria may include a predetermined duration threshold, also referred to as the duration threshold.

The capture module 318 monitors or senses potential of the ER received through the ECG electrodes 204-210, the ECG port 304, and the measurement circuit 310 after the delivery of the first pacing pulse 118. In some embodiments, the monitoring begins after the blanking period following the delivery of the first pacing pulse 118. During the blanking period, the capture module 318 disregards the sensed ER potential for a predetermined time-period to ignore residual polarization effects that may occur after the delivery of the first pacing pulse 118. In some embodiments, the capture module 318 inhibits sensing of the ECG signals 232-242 during the blanking period. In some embodiments, the blanking period is set at 40 milliseconds (ms) or may be set to a range from about 20 ms to about 80 ms.

After the blanking period, the capture module 318 begins sensing the second set of ECG signals 232-242 for a sensing period within an ER sensing window, also referred to previously as the predefined time window. In some embodiments, the ER sensing window is set at 200 ms or may be set at a range, for example, from about 40 to about 400 ms. As the sensing period begins, which is a beginning of the ER sensing window, the capture module 318 determines whether the ECG signals 232-242 in the ER sensing window meet the one or more capture criteria. In some embodiments, the sensing period is set to a range from 40 ms to 200 ms. In some embodiments, the sensing period is set to a range from 100 ms to 400 ms. In some embodiments, the sensing period is set at 40 ms and the blanking period is set at 100 ms. In some embodiments, the blanking period or the refractory period is set to a range from Oms to 200 ms and the sensing window is set to a range from 200 ms to 400 ms.

If the second set of ECG signals 232-242 satisfies the one or more capture criteria, the capture module 318 considers the satisfaction of the one or more capture criteria as an indication of an ER or the capture. In some embodiments, the amplitude threshold is set to 100 micro volt (μV) and the duration threshold is set to 100 ms. So, for example, if the ECG signals 232-242 is above 100 μV for 100 ms, such as from a wide QRS complex, in the ER sensing window then the capture module 318 decides that the pacing resulted in the capture. In some embodiments, the amplitude threshold may be set to a range from about 20 μV to about 300 μV, and the duration threshold may be set to a range from about 20 ms to about 200 ms.

In some embodiments, the amplitude threshold and/or the duration threshold may be set and/or updated by the patient's doctor or other clinician, and then programmed or updated by the processor 312. In some embodiments, if a wide QRS complex appears after the first pacing pulse 118, with a duration of 100 ms and with an amplitude for example, 100 μV, greater than the amplitude threshold, then it is determined that the first pacing pulse 118 is captured. In some embodiments, the amplitude of a QRS complex, for example, 40 μV, may be greater than the amplitude threshold. In some embodiments, the amplitude threshold is set at 100 μV and the duration threshold is set to a range from 100 ms to 400 ms.

In some embodiments, the capture module 318 specifically monitors the occurrence of a T-wave 402 of the second ECG signals 232-242 in the ER sensing window. The capture module 318 is configured to disregard other artifacts of the ECG signals 232-242, such as a retrograde P-wave 404 which indicates an atrial signal. When the T-wave 402 is sensed, the capture module 318 determines if amplitude and duration of the T-wave 402 satisfies the one or more capture criteria such as the amplitude threshold and duration threshold criteria, respectively. If the T-wave 402 satisfies the one or more capture criteria, then the capture module 318 considers such a situation as the capture situation. The presence of the T-wave 402, which satisfies the one or more capture criteria, is the ER for the delivered first pacing pulse 118. In some embodiments, the capture module 318 determines only the amplitude and the duration of the T-wave 402 for determining satisfaction of the one or more capture criteria, regardless of shape or morphology of the T-wave 402, and detection of the QRS complex and corresponding width.

In some embodiments, the blanking period is followed by the refractory period with a preset duration. During the refractory period, the capture module 318 disregards artifacts of the second set of ECG signals 232-242 until the beginning of the sensing period. In some embodiments, the capture module 318 applies only the refractory period before the sensing period, without the blanking period. In an example, the refractory period is set at 100 ms followed by the sensing period set at or up to 400 ms. In some embodiments, the refractory period is set at 40 ms, and/or the blanking period is set at 100 ms. The graph 400 also includes a Left Ventricular (LV) pressure waveform 406 depicting pressure at the left ventricle of the heart 120. The LV pressure waveform 406 is aligned to the second set of ECG signals 232-242 to display the LV pressure present corresponding to the second set of ECG signals 232-242.

If the capture is detected by the capture module 318, the pacing module 316 may maintain the pacing with same pacing parameters. The capture module 318 may continue to monitor the pacing to ensure that the capture is being maintained by the subsequent pacing pulses 118.

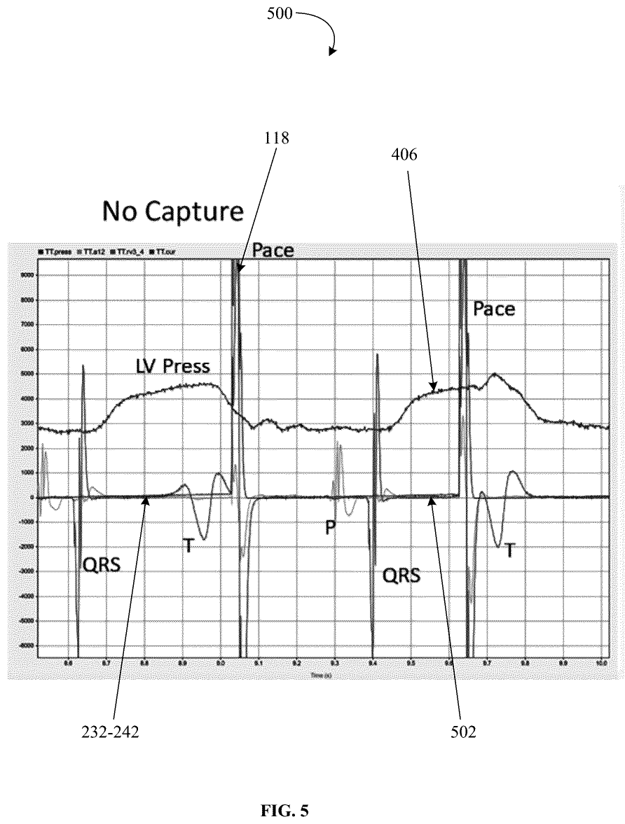

FIG. 5 illustrates a graph 500 depicting a pacing pulse no-capture scenario, according to an embodiment of the present disclosure. The graph 500 includes the first pacing pulse 118 provided during the pacing reception phase. FIG. 5 is described in conjunction with the previous figures. The second set of ECG signals 232-242 depicts a flat line 502 indicating an absence of a T-wave after providing the first pacing pulse 118. The capture module 318 considers the absence of the T-wave as the pacing pulse no-capture scenario. In some embodiments, the capture module 318 considers the presence of unwanted artifacts as the pacing pulse no-capture scenario. Upon determining that the first pacing pulse 118 did not result in the capture, the pacing module 316 changes one or more parameters of the second pacing pulse 118, for example, energy level, current level, and the like, in order to obtain the capture.

In some embodiments, the ECG signals 232-242 are filtered using one or more filters to remove unwanted artifacts of the ECG signals 232-242. The filters may include a low pass filter, a high pass filter, a bandpass filter, and the like. The unwanted artifacts include noise, the retrograde P-wave 404, and the like. For example, a notch filter may be utilized for the elimination of the high-frequency noise. Upon filtering, the filtered ECG signals are provided to the detection module 314 and the capture module 318. The filtered ECG signals are modified such that the amplitude and/or width of the filtered ECG signals are different from unfiltered ECG signals, such as the ECG signals 232-242. Hence, the processor 312 modifies the thresholds, such as the amplitude threshold and/or the duration threshold, that allows the detection and measurement of parameters corresponding to one or more artifacts, such as a T-wave, of the filtered ECG signal. For example, the amplitude threshold is modified to be set at 40 μV from 100 μV.

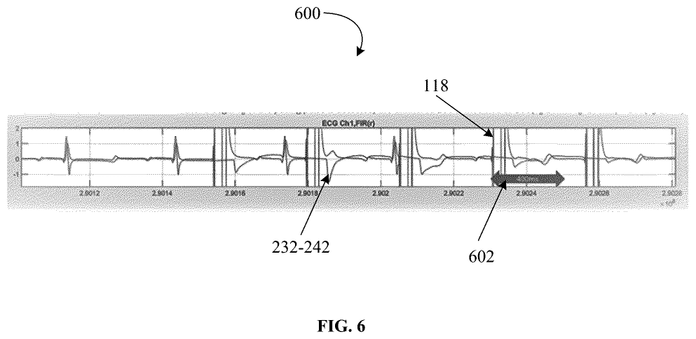

FIG. 6 illustrates a graph 600 depicting an application of the FIR filter on the ECG signals 232-242, according to an embodiment of the present disclosure. FIG. 6 is described in conjunction with the previous figures. The graph 600 includes a total period 602 that is a combination of a blanking period, a refractory period, and a sensing period. The FIR filter is a digital filter with a 16-sample difference and 10-sample average with a delay of about 40 ms when the sampling rate is 500 Hz. With a notching of about 50 Hz to 60 Hz, the FIR filter eliminates artifacts that are unnecessary for the capture analysis or determination. The FIR filter blocks or eliminates the unnecessary artifacts after application of the pacing pulse 118 resulting in creation of the blanking period or a delay of about, for example, 40 ms, without any explicit blanking of the sensing channels A, B, C, D, E, and F of the ECG electrodes 204-210. Duration of the blanking period is a design choice and may be varied based on parameters corresponding to the FIR filter. The ECG signals 232-242 are held at beginning of the blanking period until end of the blanking period. The usage of the FIR filter restricts spreading out of artifacts generated due to the pacing, which are not necessary for the capture detection, beyond duration of the FIR filter, thereby eliminating the unnecessary artifacts.

The blanking period may be set at, in some embodiments, 80 ms or 100 ms, after which the EPD 108 may then apply the refractory period. The blanking period applied due to the FIR filter is a software blanking period. The FIR filter operates during the refractory period and the sensing period after the delivery of the one or more pacing pulses 118 and the blanking period. The blanking period, in some embodiments, is similar to the predetermined time period disclosed in the previous figures. The refractory period, in some embodiments, is similar to the refractory period disclosed in the previous figures.

An external pacemaker such as the EPD 108 is aware of the time instant when the one of the one or more pacing pulses 118 has been delivered. The EPD 108 may also stop processing the ECG signals 232-242 and hold value before the pacing for a period, for example 40 ms, and then continue the processing from the previously saved value. Therefore, the FIR filter may inherently implement the blanking period thereby avoiding an explicit implementation of the blanking period for an electrical artifact from the one or more pacing pulses 118. The usage of the FIR filter enables continued filtering of the ECG signals 232-242 through an electrical artifact period, such as a period where a T-wave would occur, and blank for the refractory period. After the application of the blanking period and the refractory period, the capture module 318 senses the artifacts of the ECG signals 232-242 and determines the capture during the sensing period. The sensing period, in some embodiments, is similar to the predefined time window disclosed in the previous figures.

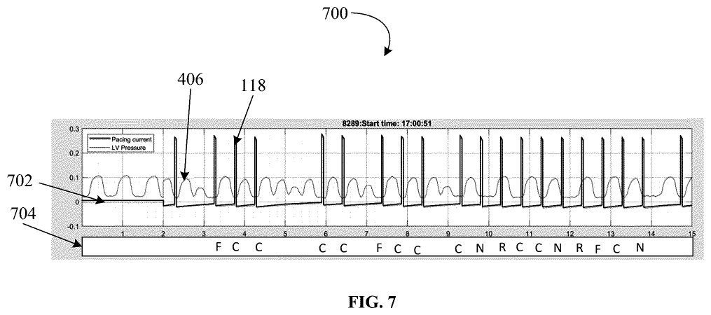

FIG. 7 illustrates a graph 700 with waveforms from the ECG electrodes 204-210, according to an embodiment of the present disclosure. FIG. 7 is described in conjunction with the previous figures.

The graph 700 illustrates the LV pressure waveform 406 and a pacing current waveform 702. After each instance of the delivery of the one or more pacing pulses 118 in the pacing current waveform 702, the capture module 318 determines whether the each of the one or more pacing pulses 118 resulted in the capture. Further, the capture module 318 or the pacing module 316 classifies the phase during which the one or more pacing pulses 118 are delivered, based on the LV pressure waveform 406. A portion 704 indicates the classification of the phases and the scenarios. The scenarios include the capture scenario and the no-capture scenario, and the phases include the refractory period phase and the fusion beat phase. The portion 704 indicates the scenarios and the phases using one of alphabets, alphanumeric characters, colors, symbols, and the like. For example, the portion 704 utilizes alphabets such as “C” for indicating the capture scenario, “N” for indicating the no-capture scenario, “F” indicates the fusion beat phase, and “R” indicates the refractory period phase.

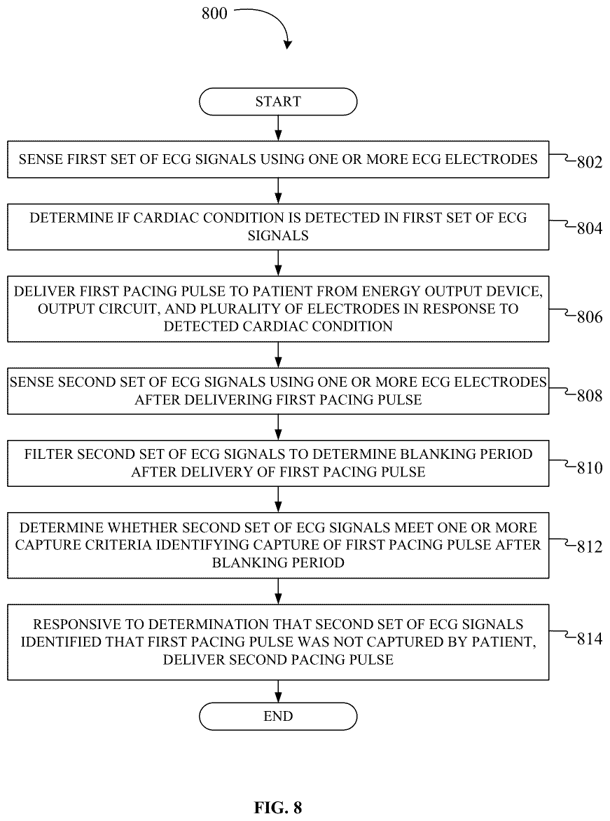

FIG. 8 illustrates an example method 800 for delivering and verifying the capture of pacing pulses using the WMS 100, according to an embodiment of the present disclosure. FIG. 8 is described in conjunction with the previous figures. In an embodiment, the method 800 is executed by the WMS 100 or the EPD 108, which may include the pacing module 316 and the capture module 318 for delivering and verifying the pacing pulses. Although the example method 800 depicts a particular sequence of operations, the sequence may be altered without departing from the scope of the present disclosure. For example, some of the operations depicted may be performed in parallel or in a different sequence that does not materially affect the function of the method 800. In other examples, different components of an example device or system that implements the method 800 may perform functions at substantially the same time or in a specific sequence.

The method 800 begins, at block 802, by sensing a first set of ECG signals using one or more ECG electrodes. In some embodiments, the EPD 108 may sense the first set of ECG signals 232-242 using the ECG electrodes 204-210 attached to or positioned on the patient 102.

Further, based on the sensed first set of ECG signals, the method 800, at block 804, includes determining if a cardiac condition is detected in the first set of ECG signals. In some embodiments, the EPD 108 determines if one of the cardiac conditions exists in the first set of ECG signals 232-242. The cardiac conditions may include the VT, VF, bradyarrhythmia or bradycardia, asystole, and the like. The EPD 108 may include one or more modules for detecting each of the cardiac conditions. After detecting the cardiac condition such as the VF, bradycardia, or the asystole, the one or more modules of the processor 312, such as the advice module 320, may provide an advice to provide the one or more pacing pulses 118.

Based on the determination, the method 800, at block 806, includes delivering a first pacing pulse to a patient from an energy output device, an output circuit, and a plurality of electrodes. In some embodiments, the EPD 108 delivers the first pacing pulse 118 to the heart 120 of the patient 102 upon detecting the cardiac condition such as the VF, bradycardia, or asystole. The delivery of the first pacing pulse 118 may be conducted using the components of the EPD 108 or the WMS 100, such as the energy output device 326, the output circuit 328, and the plurality of electrodes such as the defibrillation electrodes 112 and 114. Alternatively, in some embodiments, the delivery of the first pacing pulse 118 may be conducted using the components of the EPD 108 or the WMS 100 such as the pacing circuit 340, the output circuit 328, and the plurality of electrodes such as the defibrillation electrodes 112 and 114.

Further, the method 800, at block 808, includes sensing a second set of ECG signals using the one or more ECG electrodes after delivering the first pacing pulse. In some embodiments, the EPD 108 senses the second set of ECG signals 232-242 using the ECG electrodes 204-210 after the first pacing pulse 118 is delivered to the patient 102.

Subsequently, the method 800, at block 810, includes filtering the second set of ECG signals to determine a blanking period after delivering the first pacing pulse. In some embodiments, the EPD 108 filters the second set of ECG signals 232-242 to determine the blanking period after the delivery of the first pacing pulse 118. The EPD 108 implements the blanking period by utilizing a digital filter, such as the FIR filter. The second set of ECG signals 232-242 is filtered using the digital filter to remove unwanted artifacts of the second set of ECG signals 232-242 without any explicit blanking of the sensing channels A, B, C, D, E, and F of the ECG electrodes 204-210. The usage of the FIR filter restricts spreading out of artifacts generated due to the pacing which are not necessary and beyond duration of the FIR filter, thereby eliminating the unnecessary artifacts. In some embodiments, the usage of the DC coupled ECG electrodes 204-210 may be used for the purpose of implementing or determining the blanking period. The blanking period may be set to about 20 milliseconds to about 80 milliseconds.

The method 800, at block 812, further includes determining whether the second set of ECG signals meets one or more capture criteria identifying a capture of the first pacing pulse after the blanking period. In some embodiments, the EPD 108 identifies the capture of the first pacing pulse 118 after the blanking period by determining whether the second set of ECG signals 232-242 meets the one or more capture criteria. The EPD 108 monitors the second set of ECG signals 232-242 for the predefined time window after the blanking period. The second set of ECG signals 232-242 sensed during the predefined time window is compared with the one or more capture criteria to determine whether the capture has occurred. The one or more capture criteria may include the second set of ECG signals 232-242 reaching the predetermined ECG amplitude threshold and remaining at or above the predetermined amplitude threshold for the minimum predetermined amount of time, such as the duration threshold.

Additionally, the method 800 includes determining whether the second set of ECG signals meets the one or more capture criteria identifying an evoked response after the blanking period. An absence of the evoked response identifies that the first pacing pulse was not captured by the patient, delivering the second pacing pulse. In some embodiments, the EPD 108 determines whether the second set of ECG signals 232-242 meets the one or more capture criteria. In response to a determination that the second set of ECG signals 232-242 did not meet the one or more capture criteria, the second pacing pulse 118 is delivered. On the other hand, lack of the ER indicates that the first pacing pulse 118 was not captured by the patient 102 and the EPD 108 delivers the second pacing pulse 118.

The method 800, at block 814, further includes delivering the second pacing pulse in response to the determination that the second set of ECG signals identified that the first pacing pulse was not captured by patient. The second pacing pulse is delivered with the same pacing parameters as the first pacing pulse. In some embodiments, the EPD 108 delivers the second pacing pulse 118 to the patient 102 upon determining that the first pacing pulse 118 was not captured by the patient 102 based on the second set of ECG signals 232-242. The method 800 may include delivering the back-up pacing or the incremental pacing when the capture of the first pacing pulse 118 is not identified. On the other hand, when the first pacing pulse 118 was captured by the patient 102, the second pacing pulse 118 is not delivered to the patient 102 by the EPD 108.

Additionally, the method 800 includes using audio and/or visual alerts on a user interface to alert a bystander of at least one of: the first pacing pulse delivered to the patient, the second pacing pulse delivered to the patient, or a pacing capture status. In some embodiments, the EPD 108 utilizes the UI 306 for providing the audio and/or visual alerts to alert the bystander 102 of at least one of: the first pacing pulse 118 delivered to the patient 102, the second pacing pulse 118 delivered to the patient 102, or the pacing capture status. Any module of the EPD 108 or any component of the WMS 100 may perform each or at least one process disclosed in the one or more blocks 802-814 of the method 800.

The WMS 100, along with the corresponding method 800, is utilized for a non-invasive process of detecting cardiac rhythm disorders, providing therapy to a patient based on the detected cardiac rhythm disorders, and verifying or determining the success of the provided therapy. The WMS 100 is a single mechanism which is capable of detecting different cardiac rhythm disorders such as VF, bradyarrhythmia, and/or asystole, and providing therapy by, for example, providing pacing pulses. Further, the WMS 100 also verifies if the provided pacing pulses have resulted in a capture.

Upon determining that the provided pacing pulses have not resulted in the capture, the WMS 100 continues to provide subsequent pacing pulses as resuscitation attempts. In parallel, the WMS 100 notifies the patient wearing the WMS 100 and also one or more bystanders close to or in contact with the patient regarding an impending pacing or defibrillation shock. This allows the patient to determine if the impending pacing or the defibrillation shock is erroneous and provides an input for aborting the pacing or the defibrillation shock. Also, one or more alerts or notifications allow the bystanders to move away from the patient or avoid interfering with the delivery of the pacing pulses to avoid experiencing painful pacing pulses due to conduction of the pacing pulses through the bystander. The WMS 100 communicates regarding the delivery of the pacing pulses to an emergency responder.

Further, the WMS 100 also notifies or alerts regarding the capture status of the pacing pulses and corresponding parameters, such as energy or current level, to the patient, the bystander, and the emergency responders. Based on the capture status, the patient, the bystander, or the emergency responders may determine a next course of action which may include the CPR or other resuscitation attempts if the capture status indicates a failure.

Other embodiments include combinations and sub-combinations of features described or shown in the drawings herein, including for example, embodiments that are equivalent to: providing or applying a feature in a different order than in a described embodiment, extracting an individual feature from one embodiment and inserting such feature into another embodiment; removing one or more features from an embodiment; or both removing one or more features from an embodiment and adding one or more features extracted from one or more other embodiments, while providing the advantages of the features incorporated in such combinations and sub-combinations. As used in this paragraph, feature or features can refer to the structures and/or functions of an apparatus, article of manufacture or system, and/or the steps, acts, or modalities of a method.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A wearable medical system, comprising:
a support structure configured to be worn by a patient;
a plurality of ECG electrodes to sense an ECG signal of the patient;
an energy output device to store electrical charge;
an output circuit coupled to the energy output device;
a plurality of therapy electrodes engaged to the support structure and in communication with the output circuit, to deliver therapy to the patient; and
a processor, in communication with the plurality of ECG electrodes and the output circuit, the processor configured to:
cause a pacing pulse to be delivered to the patient via the energy output device, the output circuit, and the plurality of therapy electrodes,
determine whether the pacing pulse was captured by determining whether the ECG signal, within a window subsequent to a blanking period and a refractory period following delivery of the pacing pulse, meets one or more capture criteria,
responsive to a determination that the pacing pulse was captured, prevent an additional pacing pulse from being delivered to the patient, and
responsive to a determination that the pacing pulse was not captured, cause an additional pacing pulse to be delivered to the patient.

2. The wearable medical system of claim 1, wherein the one or more capture criteria comprise an ECG amplitude threshold and a duration threshold, wherein the duration threshold is met when an ECG amplitude measured within the window is above the ECG amplitude threshold for a predetermined minimum amount of time.

3. The wearable medical system of claim 1, wherein the plurality of ECG electrodes are resistive, DC-coupled ECG electrodes.

4. The wearable medical system of claim 1, wherein the blanking period is implemented by a finite impulse response filter.

5. The wearable medical system of claim 4, wherein the finite impulse response filter continues filtering ECG signals during an electrical artifact period.

6. The wearable medical system of claim 1, further comprising a user interface configured to alert bystanders through audio and/or visual alerts of at least one of: the pacing pulse being delivered to the patient and a pacing capture status.

7. A method for delivering and verifying pacing pulses, the method comprising:
sensing a first set of ECG signals using one or more ECG electrodes;
determining if a cardiac condition is detected in the first set of ECG signals;
delivering a first pacing pulse to a patient from an energy output device, an output circuit, and a plurality of electrodes in response to a detected cardiac condition;
sensing a second set of ECG signals using the one or more ECG electrodes after delivering the first pacing pulse;
filtering the second set of ECG signals to determine a blanking period after delivering the first pacing pulse;
determining whether the second set of ECG signals meet one or more capture criteria identifying a capture of the first pacing pulse after the blanking period; and
wherein, responsive to a determination that the second set of ECG signals identified that the first pacing pulse was not captured by the patient, delivering a second pacing pulse.

8. The method of claim 7, further comprising:
determining whether the second set of ECG signals meet the one or more capture criteria identifying an evoked response after the blanking period; and
wherein, responsive to a determination that the second set of ECG signals did not meet the one or more capture criteria, delivering the second pacing pulse.

9. The method of claim 7, wherein the capture of the first pacing pulse is identified responsive to a determination that the second set of ECG signals exceed at least one of a voltage threshold or a duration threshold.

10. The method of claim 7, wherein the cardiac condition is bradycardia and/or asystole.

11. The method claim 7, wherein the second pacing pulse is delivered with the same pacing parameters as the first pacing pulse.

12. The method of claim 7, further comprising delivering a back-up pacing or an incremental pacing responsive to a determination that the second set of ECG signals identified that the first pacing pulse was not captured by the patient.

13. The method of claim 7, wherein the blanking period is about 20 milliseconds to about 80 milliseconds.

14. The method of claim 7, further comprising:
using audio and/or visual alerts on a user interface to alert a bystander of at least one of: the first pacing pulse delivered to the patient, the second pacing pulse delivered to the patient, or a pacing capture status.

15. A non-transitory computer-readable medium, encoded with instructions for delivering and verifying capture of a pacing pulse by a patient stored thereon that, when executed by a computing device cause the computing device to perform operations for delivering and verifying capture of the pacing pulse by the patient, the operations comprising:
receiving a first set of ECG signals of the patient via one or more ECG electrodes;
determining, by a cardiac condition detector, if the first set of ECG signals are indicative of a cardiac condition treatable by pacing;
activating an energy output device, output circuit, and plurality of therapy electrodes to deliver a first pacing pulse to the patient in response to a determination that the first set of ECG signals are indicative of the cardiac condition;
receiving a second set of ECG signals of the patient via one or more ECG electrodes;
determining, using a digital filter, a blanking period that begins after the first pacing pulse is delivered;
determining, by a capture module, if the second set of ECG signals meet one or more capture criteria indicative of a capture of the first pacing pulse after the blanking period; and
responsive to a determination by the capture module that the second set of ECG signals are not indicative of capture of the first pacing pulse, delivering a second pacing pulse using the energy output device, the output circuit, and the plurality of therapy electrodes.

16. The non-transitory computer-readable medium of claim 15, wherein the capture module determines that the second set of ECG signals are indicative of the capture of the first pacing pulse in response to the second set of ECG signals exceeding at least one of a voltage threshold or a duration threshold.

17. The non-transitory computer-readable medium of claim 15, wherein the cardiac condition is bradycardia and/or asystole.

18. The non-transitory computer-readable medium of claim 15, wherein the second pacing pulse is a back-up pacing pulse or an incremental pacing pulse.

19. The non-transitory computer-readable medium of claim 15, wherein the blanking period is about 20 milliseconds to about 80 milliseconds.

20. The non-transitory computer-readable medium of claim 15, wherein the operations further comprising:
generating, by a user interface, audio and/or visual alerts to alert a bystander of at least one of the first pacing pulse delivered to the patient, the second pacing pulse delivered to the patient, or a pacing capture status.
